(19) United States
(12) Patent Application Publication
(10) Pub. No.: US 2025/0354958 A1
(43) Pub. Date: Nov. 20, 2025

(54) GAS SAMPLE EVALUATION DEVICE FOR GRAPHENE SENSOR ARRAYS

(71) Applicant: VOCxI Health, Inc., Arden Hills, MN (US)

(72) Inventors: Gregory J. Sherwood, White Bear Lake, MN (US); Raia Colette Finc, Shoreview, MN (US); Gregory Kermit Peterson, Centerville, MN (US); Yung-Ping Yeh, Eden Prairie, MN (US); Randall Lee Schiestl, Brooklyn Park, MN (US)

(21) Appl. No.: 19/195,957

(22) Filed: May 1, 2025

Related U.S. Application Data
(60) Provisional application No. 63/641,692, filed on May 2, 2024.

(51) Int. Cl. G01N 27/60; G01N 1/22

(52) U.S. Cl. CPC G01N 27/60; G01N 1/2247; G01N 2001/2244

(57) ABSTRACT

Embodiments herein relate to systems and devices for evaluating gas samples. In an embodiment a measurement system for gas samples can be included having a housing defining an inflow port and an outflow port and including a sensor board, wherein the sensor board can be disposed within the housing. The sensor board can include a first side and a second side. The measurement system for gas samples can define a flow path, wherein the flow path extends from the inflow port to the outflow port. A plurality of graphene sensors can be disposed on the second side. Other embodiments are also included herein.

This application claims the benefit of U.S. Provisional Application No. 63/641,692, filed May 2, 2024, the content of which is herein incorporated by reference in its entirety.

### FIELD

Embodiments herein relate to systems and devices for evaluating gas samples.

### BACKGROUND

The accurate detection of diseases can allow clinicians and others to provide appropriate therapeutic and other interventions. The early detection of diseases can lead to better treatment outcomes. Diseases can be detected using many different techniques including analyzing tissue samples, analyzing various bodily fluids, diagnostic scans, genetic sequencing, and the like.

Many disease states result in the production of specific chemical compounds. In some cases, volatile organic compounds (VOCs) released into a gaseous sample of a patient or subject can be hallmarks of certain diseases or conditions. The detection of these compounds or differential sensing of the same can allow for the early detection of particular disease states. Similarly, beyond detection of disease states, various compounds and/or their metabolites can be contained within gaseous samples from a patient or subject. Detection of such compounds can provide information regarding the state or condition of the patient or subject or sample.

### SUMMARY

Embodiments herein relate to systems and devices for evaluating gas samples. In a first aspect, a measurement system for gas samples can be included having a housing defining an inflow port and an outflow port and including a sensor board, wherein the sensor board can be disposed within the housing. The sensor board can include a first side and a second side. The measurement system for gas samples can define a flow path, wherein the flow path extends from the inflow port to the outflow port. A plurality of graphene sensors can be disposed on the second side.

In a second aspect, in addition to one or more of the preceding or following aspects, or in the alternative to some aspects, the sensor board can further include a circuit board.

In a third aspect, in addition to one or more of the preceding or following aspects, or in the alternative to some aspects, the graphene sensors can include graphene varactors.

In a fourth aspect, in addition to one or more of the preceding or following aspects, or in the alternative to some aspects, the flow path passes along and can be in contact with a lengthwise axis of the first side of the sensor board.

In a fifth aspect, in addition to one or more of the preceding or following aspects, or in the alternative to some aspects, the first side of the sensor board can be configured to allow condensation of moisture thereon reducing the humidity of an incoming gas sample.

In a sixth aspect, in addition to one or more of the preceding or following aspects, or in the alternative to some aspects, can further include a control circuit, wherein the control circuit can be configured to control operations of the measurement system for gas samples.

In a seventh aspect, in addition to one or more of the preceding or following aspects, or in the alternative to some aspects, can further include a power supply.

In an eighth aspect, in addition to one or more of the preceding or following aspects, or in the alternative to some aspects, the power supply can be configured to receive 500 mA or less DC current.

In a ninth aspect, in addition to one or more of the preceding or following aspects, or in the alternative to some aspects, can further include measurement circuitry, wherein the measurement circuitry can be configured to provide a stimulus voltage to the plurality of graphene sensors and measure an electrical property of the same.

In a tenth aspect, in addition to one or more of the preceding or following aspects, or in the alternative to some aspects, can further include wireless communications circuitry.

In an eleventh aspect, in addition to one or more of the preceding or following aspects, or in the alternative to some aspects, can further include wired communications circuitry.

In a twelfth aspect, in addition to one or more of the preceding or following aspects, or in the alternative to some aspects, can further include an inflow tube, wherein the inflow tube can be in fluid communication with the inflow port.

In a thirteenth aspect, in addition to one or more of the preceding or following aspects, or in the alternative to some aspects, the inflow tube can include: an inspiration port, and an expiration port, wherein the expiration port can be in fluid communication with the inflow port of the housing.

In a fourteenth aspect, in addition to one or more of the preceding or following aspects, or in the alternative to some aspects, the measurement system for gas samples can further include an inspiration valve, wherein the inspiration valve can be a one-way valve controlling movement of air through an inspiration port, and an expiration valve, wherein the expiration valve can be a one-way valve controlling movement of air through the expiration valve.

In a fifteenth aspect, in addition to one or more of the preceding or following aspects, or in the alternative to some aspects, can further include an outflow valve, wherein the outflow valve can be a one-way valve controlling movement of air through the outflow port.

In a sixteenth aspect, in addition to one or more of the preceding or following aspects, or in the alternative to some aspects, can further include at least one electrically powered heating element, wherein the at least one electrically powered heating element can be disposed on the second side of the sensor board adjacent to the plurality of graphene sensors.

In a seventeenth aspect, in addition to one or more of the preceding or following aspects, or in the alternative to some aspects, can further include a removable hermetic sealing layer or other sealing materials, wherein the removable hermetic sealing layer or other sealing materials can be disposed over the plurality of graphene sensors.

In an eighteenth aspect, in addition to one or more of the preceding or following aspects, or in the alternative to some aspects, the removable hermetic sealing layer or other sealing materials can include a metal foil layer.

In a nineteenth aspect, in addition to one or more of the preceding or following aspects, or in the alternative to some aspects, the removable hermetic sealing layer or other sealing materials seals in an inert gas against the plurality of graphene sensors.

In a twentieth aspect, in addition to one or more of the preceding or following aspects, or in the alternative to some aspects, the measurement system for gas samples can be a handheld system.

In a twenty-first aspect, in addition to one or more of the preceding or following aspects, or in the alternative to some aspects, the housing can include a two-piece clam shell.

In a twenty-second aspect, in addition to one or more of the preceding or following aspects, or in the alternative to some aspects, can further include a tamper-evident seal, wherein the tamper-evident seal can be configured to maintain engagement of the pieces of the two-piece clam shell and the tamper-evident seal can be frangible to make disengagement and reengagement of the pieces of the two-piece clam shell visible.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of components of a gas sample evaluation system in accordance with various embodiments herein.

FIG. 2 is a schematic perspective view of a gas sample evaluation system housing unit in accordance with various embodiments herein.

FIG. 3 is a schematic perspective view of a gas sample evaluation system housing unit in accordance with various embodiments herein.

FIG. 4 is an exploded view of a gas sample evaluation system housing unit and components therein in accordance with various embodiments herein.

FIG. 5 is a sectional view of a gas sample evaluation system housing unit and components therein in accordance with various embodiments herein.

FIG. 6 is a schematic view of a side of a sensor board in accordance with various embodiments herein.

FIG. 7 is a schematic view of a side of a sensor board in accordance with various embodiments herein.

FIG. 8 is a sectional view of a portion of a sensor board in accordance with various embodiments herein.

FIG. 9 is a schematic view of a side of a sensor board in accordance with various embodiments herein.

FIG. 10 is a schematic view of a piece of a clam shell housing in accordance with various embodiments herein.

FIG. 11 is a schematic view of a piece of a clam shell housing in accordance with various embodiments herein.

FIG. 12 is a schematic view of a piece of a clam shell housing in accordance with various embodiments herein.

FIG. 13 is a schematic view of a piece of a clam shell housing in accordance with various embodiments herein.

FIG. 14 is a schematic view of components of a gas sample evaluation system for gas samples in accordance with various embodiments herein.

FIG. 15 is a schematic view of a side of a sensor board in accordance with various embodiments herein.

FIG. 16 is a schematic sectional view of a portion of a device herein illustrating how a removable hermetic sealing layer can be removed.

FIG. 17 is a schematic view of various components of a system in accordance with various embodiments herein.

FIG. 18 is a schematic diagram of a portion of a measurement zone in accordance with various embodiments herein.

FIG. 19 is a schematic perspective view of a graphene varactor in accordance with various embodiments herein.

FIG. 20 is a schematic cross-sectional view of a portion of a graphene varactor in accordance with various embodiments herein.

FIG. 21 is a schematic diagram of an example of measurement circuity to measure the capacitance of graphene sensors in accordance with various embodiments herein.

FIG. 22 is a schematic perspective view of a portion of a gas sample evaluation system housing unit in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Embodiments herein include gas sample evaluation devices and systems. Applications for embodiments herein include human, animal, instrumentation, environmental, and other measurements/detections to include but not limited to injury, disease, toxins, drugs, and chemicals.

In some embodiments, a gas sample evaluation system herein can include a housing around a circuit board that directs an incoming flow of a gas sample across one side of the circuit board, around the board to the other side of the circuit board, across the second side of the circuit board and then across an array of graphene sensors. In this way, the design reduces the possibility of condensation forming on the surface of the graphene sensors, which can inhibit their ability to function accurately. Furthermore, in various embodiments herein, one or more thermal conductors can be included on the circuit board to convey heat from the incoming gas sample from one side of the circuit board to the other side that carries the plurality of graphene sensors. In this way, heat from the gas sample can be used to warm the area of the graphene sensors to a point above the dew point for the gas sample and prevent condensation from occurring while also minimizing the amount of heat that may need to be generated using powered heat sources. Thus, the amount of power needed by the system can be reduced and can facilitate powering the system with may be available from a mobile device such as a tablet or phone or battery thereby enhancing the portability of the system.

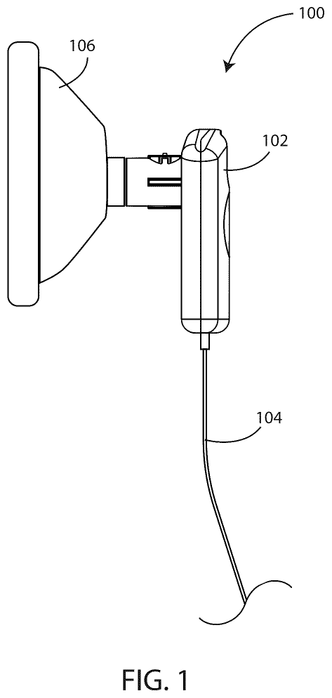

Referring now to FIG. 1, a schematic view of components of a gas sample evaluation/measurement system is shown in accordance with various embodiments herein. The system includes a main housing 102. Various components, including graphene-based sensors, can be disposed within the main sensor housing 102 and will be described in greater depth below. A mask 106 can be connected to the main housing 102, such as to facilitate gathering a gas sample from an individual. However, it will be appreciated that there are various ways of gathering gas samples and a mask 106 can be omitted in various embodiments. In this embodiment, the system also includes a wired connection 104, such as to provide power, transmit data, or the like. In some embodiments, the wired connection 104 can be a USB cable connected to a separate device (such as a phone, tablet computing device, computer, or the like). In various embodiments, a power supply of the system can be configured to receive 500 mA or less DC current, such as through the wired connection 104.

It has been found that with various configurations herein, using 500 mA (a limitation of some mobile devices) can require significant time to heat sensors herein to a desire temperature. For example, it can require 3 minutes to heat the sensors to approximately 26 degrees Celsius. To allow for more rapid heating, in some embodiments herein, a battery and/or a capacitor can be included and can be used to provide energy to one or more heating elements to allow for a more rapid heating process that can allow the system to take measurements more quickly.

In some embodiments, the system can communicate through wired or wireless means and can include wired and/or wireless data communication components such as an antenna, transmitter, receiver, transceiver, or the like. Wireless communications can be executed according to various protocols including but not limited to WIFI (802.11ax, ac, n, g, b, etc.), BLUETOOTH, ZIGBEE, Z-Wave, LTE, 5G protocols, and the like.

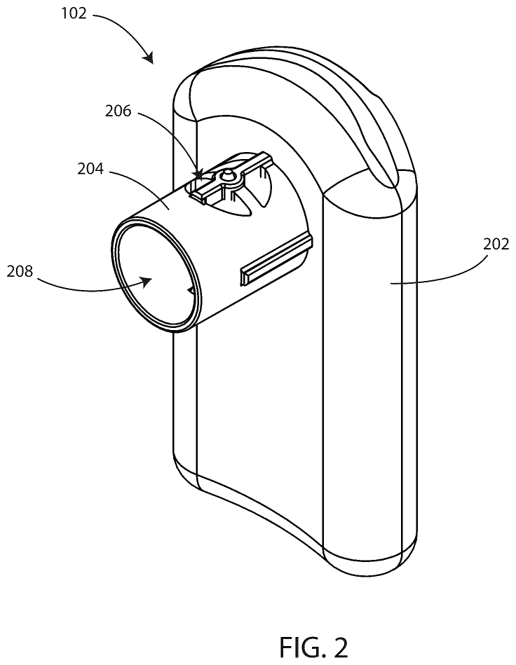

Referring now to FIG. 2, a schematic view of a gas sample evaluation system housing 102 is shown in accordance with various embodiments herein. In this embodiment, the housing 102 takes the form of a two-piece clam shell 202. However, it will be appreciated that other forms are also contemplated herein. In this embodiment, the measurement system for gas samples also includes an inflow tube 204. The inflow tube 204 defines a gas sample inflow port 208 as well as an inspiration port 206. In use, a subject can first breathe in causing air to be pulled in from the ambient environment through the inspiration port 206 through the inflow tube 204 and out the gas sample inflow port 208 to their lungs. Then they can exhale the gas sample which flows through the gas sample inflow port 208 and into the housing 102 of the system.

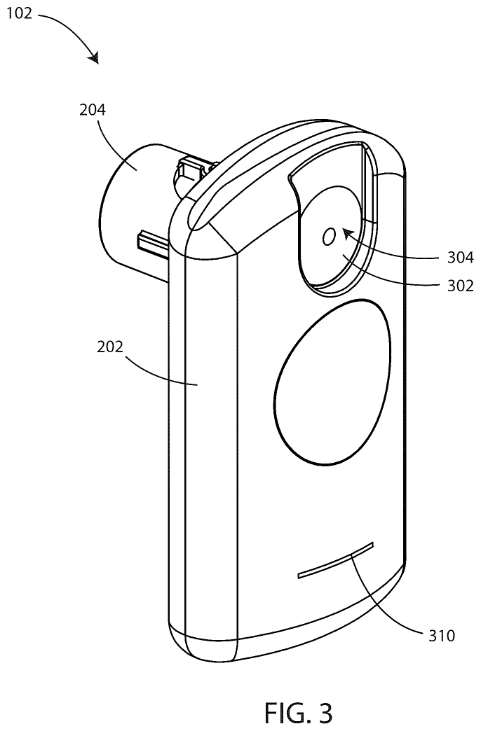

Referring now to FIG. 3, a schematic view of a gas sample evaluation system housing 102 unit is shown in accordance with various embodiments herein. In this view, the housing 102 also includes an outflow port 304. After a gas sample flows past the graphene sensors, it can flow out of the outflow port 304. An outflow valve 302 can be included, such as a one-way valve, to prevent air from flowing into the housing 102 through the outflow port 304. The housing 102 can also include a slot 310 therein, through which the pull tab (described below) can extend.

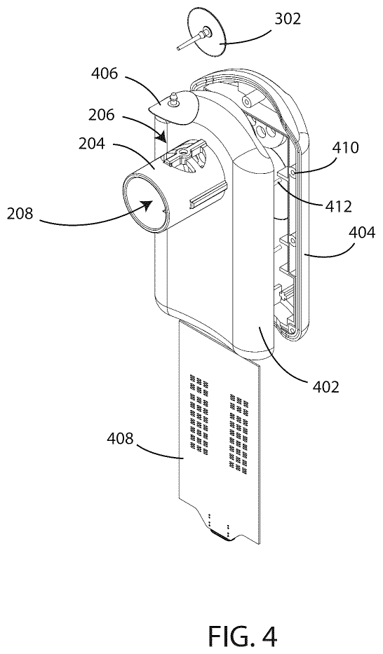

Referring now to FIG. 4, an exploded view of a gas sample evaluation system housing 102 and components therein is shown in accordance with various embodiments herein. In this example, the housing 102 is a two-piece clam shell configuration including a first piece 402 and a second piece 404. Pieces of the housing can be formed of polymers (such as a thermoplastic polymer or a thermoset polymer), metals, composites, or the like. Pieces of the housing can be formed in various ways. In some embodiments, pieces of the housing are formed using injection molding techniques. In some embodiments, pieces of the housing can be formed using additive printing techniques.

In this embodiment, the two-piece clam shell includes guide holes 410 and guide pegs 412. The guide pegs 412 can be configured to fit within the guide holes 410 to facilitate proper alignment of the claim shell pieces when they are assembled. The housing also defines an inflow port 208.

In this view, an inspiration port valve 406 is shown. The inspiration port valve 406 can be a one-way valve to prevent air from flowing out of the inspiration port 206. The measurement system for gas samples also includes a sensor board 408 or circuit board disposed inside the clam shell structure. Graphene sensors can be disposed on the sensor board 408 along with other components (such as thermal conductors) as described more fully below.

In some embodiments, the guide holes 410 can extend to an outside surface of a housing 102 to facilitate separation of the first piece 402 of the housing 102 from the second piece 404 of the housing 102, such as to facilitate removal of the sensor board 408. For example, one or more disassembly pins can be inserted into the guide holes 410 from the outside of the housing 102 causing the guide pegs 412 to be pushed out of the guide holes 410 and allowing the housing 102 to be taken apart.

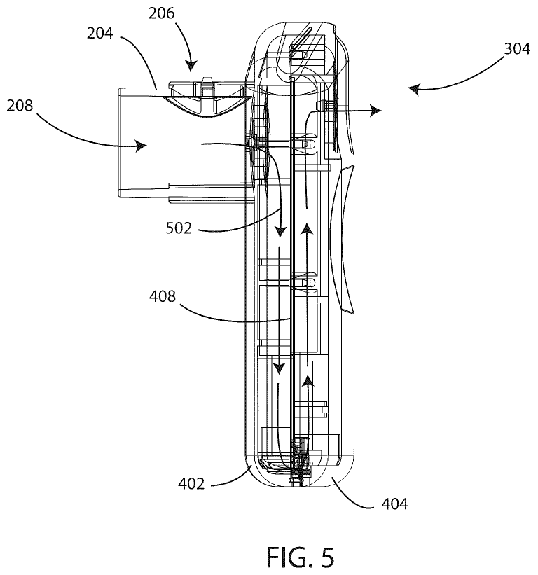

Referring now to FIG. 5, a sectional view of a gas sample evaluation system housing 102 unit and components therein is shown in accordance with various embodiments herein. As before, a two-piece clam shell configuration of the housing includes a first piece 402 and a second piece 404. The housing defines an inflow port 208 through which a gas sample from a subject passes. The gas sample follows a flow path 502 as illustrated by the arrows in FIG. 5 extending from the inflow port 208 to an outflow port 304. In specific, the flow path 502 passes across one side of the sensor board 408, around the board 408 to the other side of the sensor board 408, across the second side of the sensor board 408, and then across an array of graphene sensors disposed on sensor board 408. As before, the housing also includes an outflow port 304 out of which the gas sample passes.

In various embodiments, the flow path 502 is a circuitous path. In various embodiments, the flow path 502 passes along and is in contact with a lengthwise axis of the first side of the sensor board 408. In various embodiments, the flow path 502 flows over a first side of a sensor board 408, which is configured to allow for condensation of moisture thereon reducing the humidity of an incoming gas sample.

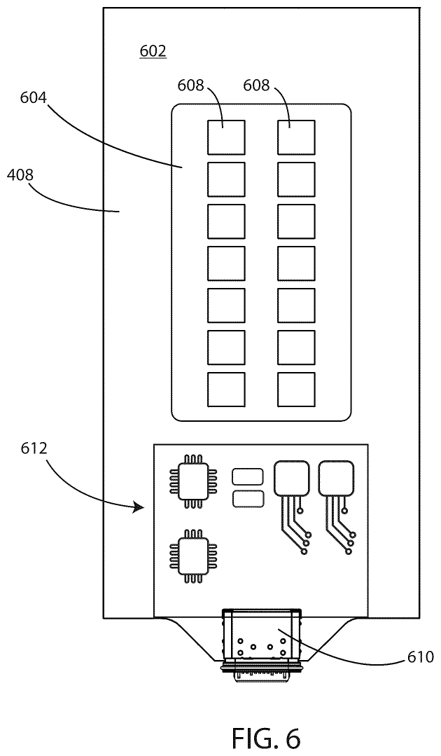

Referring now to FIG. 6, a schematic view is shown of a side of a sensor board 408 in accordance with various embodiments herein. In specific, FIG. 6 shows one side 602 of the sensor board 408. The sensor board 408 includes a sensor area 604 and the sensor area 604 includes an array of graphene sensors 608. The sensor board 408 also includes electronic components area 612, which can include various electronic components described herein.

In various embodiments, the measurement system for gas samples also includes a connection port 610, which can facilitate wired communications of the system with other components and/or can provide power and can be configured to be accessible even after the pieces of the clam shell housing are attached to one another.

In various embodiments, one or more thermal conductors are disposed underneath at least some of the plurality of graphene sensors 608. In some cases, the thermal conductors are disposed underneath the sensor area 604. However, in some cases, the thermal conductors can also be disposed outside of the sensor area 604.

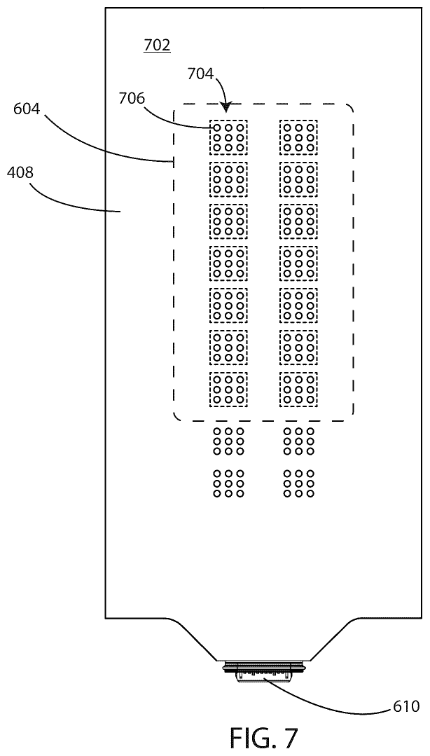

Referring now to FIG. 7, a schematic view is shown of an opposite side 702 of the sensor board 408 in accordance with various embodiments herein. The sensor area 604 from FIG. 6 of the sensor board 408 is shown in dashed lines to indicate its location. The sensor board 408 also includes one or more thermal conductors 704.

In various embodiments, the one or more thermal conductors 704 pass from a first side 702 to a second side 602 of the sensor board 408. In various embodiments, the one or more thermal conductors 704 are disposed within 3 centimeters of a plurality of graphene sensors 608. In various embodiments, the one or more thermal conductors 704 are disposed underneath at least some of a plurality of graphene sensors 608. Generally, the more surface area that is occupied by thermal conductors, the more easily heat can be conveyed from one side of the sensor board 408 to the other. In some embodiments, the one or more thermal conductors 704 take up at least about 0.5, 1, 2, 3, 4, 5, 6, 7, 8, 9 10, 15, or 20% or more of the surface area of a sensor board 408, or an amount falling within a range between any of the foregoing.

The one or more thermal conductors 704 can take various forms. In some embodiments, the thermal conductors can include one or more metal vias 706 that pass from one side of the sensor board 408 to the other. In some embodiments, the metal vias 706 can be copper, however, other metals or other conductors of heat are also contemplated herein. The metal vias can exist for the purpose of conducting heat versus for the purpose of connecting portions of a circuit. As such, in various embodiments, the metal vias are configured to not be part of an electrical circuit of the sensor board 408.

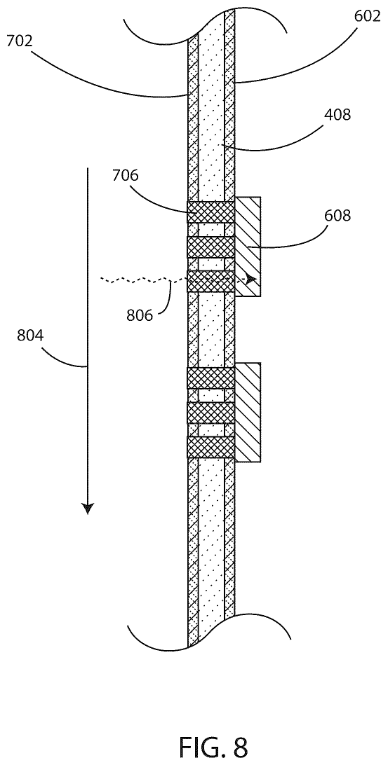

Referring now to FIG. 8, a sectional view of a portion of a sensor board 408 is shown in accordance with various embodiments herein. FIG. 8 illustrates both sides, 602 and 702, of the sensor board 408. The array of graphene sensors 608 can be seen disposed on side 602. Thermal conductors are present in the form of metal vias 706. In some embodiments, the sensor board 408 can be formed of a circuit board material, such as standard printed circuit board materials including, for example, layers of conductors (such as copper or other metal foil) and layers of insulators (such as polymers or the like).

FIG. 8 illustrates a gas sample flow path 804. The gas sample flow path 804 passes over a side 702 of the sensor board 408 and heat 806 from the gas transfers to the metal vias 706 and into the area of the graphene sensor 608. In some embodiments, in use the portions of the sensor board on side 602 in the area of the metal vias and/or the graphene sensors 608 are at least 0.5, 1, 2, 3, 4, 5 or more degrees Celsius warmer than surfaces on side 602 that are at least 1 centimeter away from any thermal conductors in the form of metal vias herein.

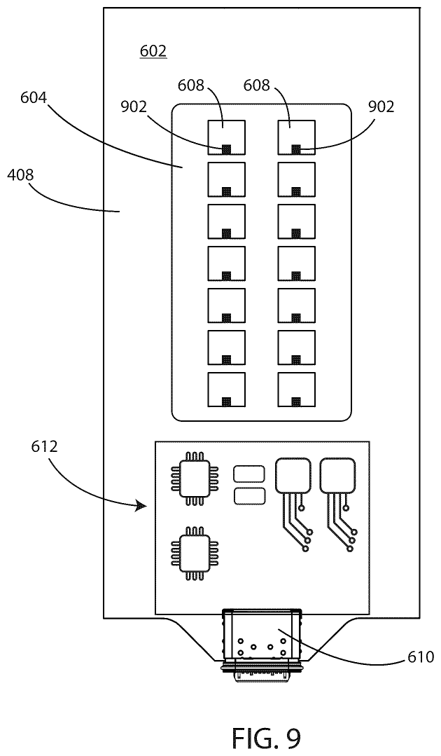

In some embodiments, one or more powered heat sources can also be used to heat an area covering and/or adjacent to the area bearing the graphene sensor. However, features herein that facilitate heat transfer from an incoming gas sample from one side of the sensor board to the other can allow for such powered heat sources to be minimized or even eliminated. Referring now to FIG. 9, a schematic view of a side of a sensor board 408 is shown in accordance with various embodiments herein. FIG. 9 is generally similar to FIG. 6 and shows a side 602 of the sensor board including a sensor area 604 and an array of graphene sensors 608 disposed within the sensor area 604 along with electronic components area 612. As before, the measurement system for gas samples is also shown with a connection port 610, although in some embodiments the connection port 610 can be omitted (such as in the case of a battery powered unit and/or wireless data transmission). In this example, the measurement system for gas samples also includes an at least one electrically powered heating element 902. The electrically powered heating element(s) 902 can be disposed adjacent to the array of graphene sensors 608. The electrically powered heating element(s) 902 can take the form of resistive type heating elements (resulting in Joule heating) or can take other forms.

In some embodiments, a sample flow path can follow a direct path between an inflow port and a point where it crosses over from one side of the sensor board to the other side of the sensor board. However, as will be illustrated further below, in some embodiments the sample flow path lacks a direct path between an inflow port and a point where it crosses over from one side to the other side of the sensor board. For example, the sample flow path can be circuitous which can increase the contact length and therefore the time in which the gas sample is in contact with a surface of sensor board so as to allow for more time for heat to transfer from the gas sample to the sensor board. In some embodiments, the flow path includes at least one segment that is perpendicular to a lengthwise axis of a sensor board 408 and falls within a plane that is parallel to a first side of the sensor board 408. In various embodiments, the gas sample flow path can be configured to cause the gas sample flowing there through to move in multiple directions with respect to a lengthwise axis of a sensor board (such as both down and up).

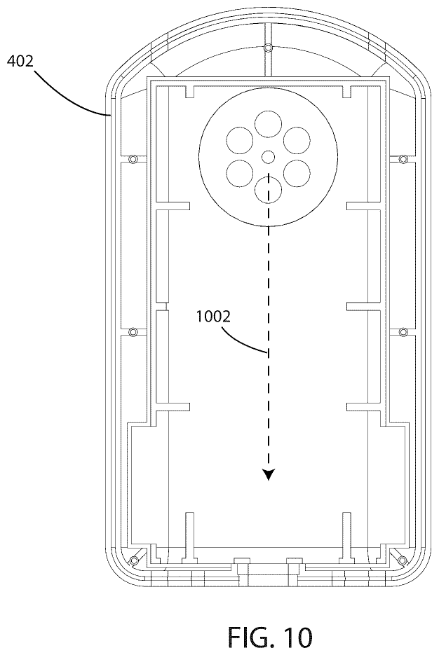

Referring now to FIG. 10, a schematic view is shown of the first piece 402 of the clam shell housing 102 in accordance with various embodiments herein. FIG. 10 also shows a sample flow path 1002, which is largely a straight line going from near the top of where the sensor board (not shown in this view) would be with respect to the first piece 402 to the near the bottom of where the sensor board would be with respect to the first piece 402. This arrangement can be used, however, baffles and/or other flow directing structures can be disposed on or over the first piece 402 of the housing and/or the sensor board itself to create a gas flow path that is longer, such as in the case of a circuitous gas flow path.

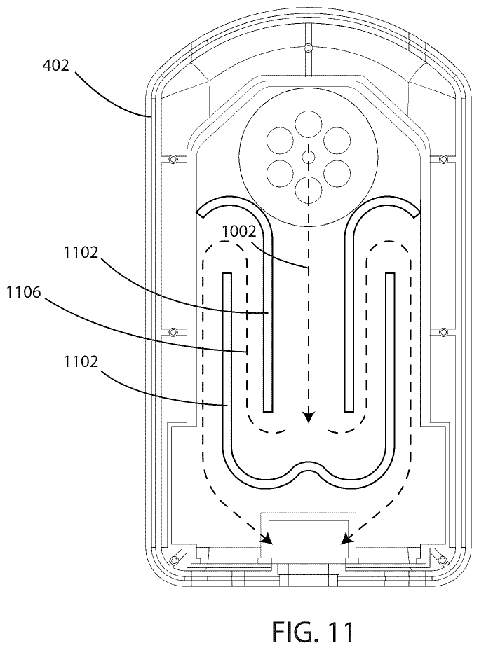

Referring now to FIG. 11, a schematic view of the first piece 402 of the clam shell housing 102 is shown in accordance with various embodiments herein. In this embodiment, the first piece 402 includes flow baffle structures 1102 or flow guides to direct the gas sample flow path 1002 to be something other than a straight line, such as a circuitous path 1106. In some embodiments, the flow baffle structures 1102 can be formed integrally with the first piece 402 of the housing 102. In some embodiments, the flow baffle structures 1102 can be formed separately from the first piece of the housing 102. In some embodiments, flow baffle structures 1102 including one or more curved portions. In some embodiments, the flow baffle structures 1102 are substantially straight.

In some embodiments, the flow path 1002 defines a circuitous path 1106 that is in contact with a side of the sensor board (such as the opposite side from the side which carries the array of graphene sensors). In various embodiments, the flow path 1002 lacks a direct path between the inflow port and a point where it crosses over from the one side of the sensor board to the other side. In some embodiments, the flow path includes at least a 90 degree or greater turn, a 120 degree or greater turn, a 150 degree or greater turn, or a 180 degree or greater turn. In some embodiments, the flow path 1002 causes air flowing there through to move in two opposite directions (such as up and down) with respect to a lengthwise axis of the sensor board 408. This can greatly lengthen the flow path allowing more time for heat transfer to occur. In some embodiments, the flow path is at least about 1.1, 1.2, 1.3, 1.4, 1.5, 1.6, 1.7, 1.8, 1.9, 2, 2.25, 2.5, 2.75, 3, or 4 times longer (or an amount falling within a range between any of the foregoing) than a flow path for a sensor board 408 of equal size where the flow path is simply straight.

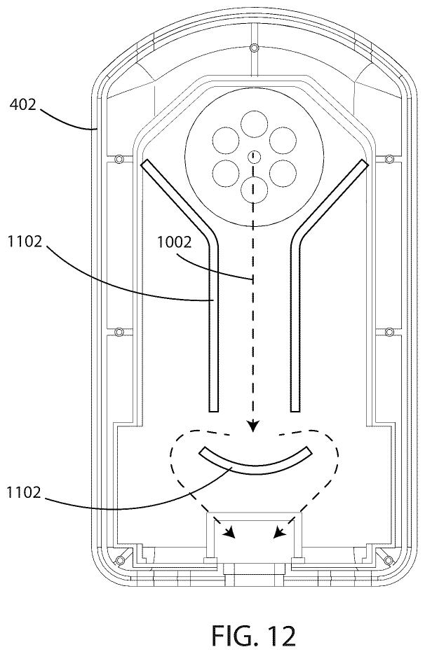

Flow paths can be configured in many different ways herein. Referring now to FIG. 12, a schematic view of a piece 402 of a clam shell housing 102 is shown in accordance with various embodiments herein. As before, the first piece 402 includes flow baffles 1102 or flow guides. However, the flow baffles 1102 are different in shape than those illustrated in FIG. 11. As such, the flow path 1002 in FIG. 12 is different than in FIG. 11.

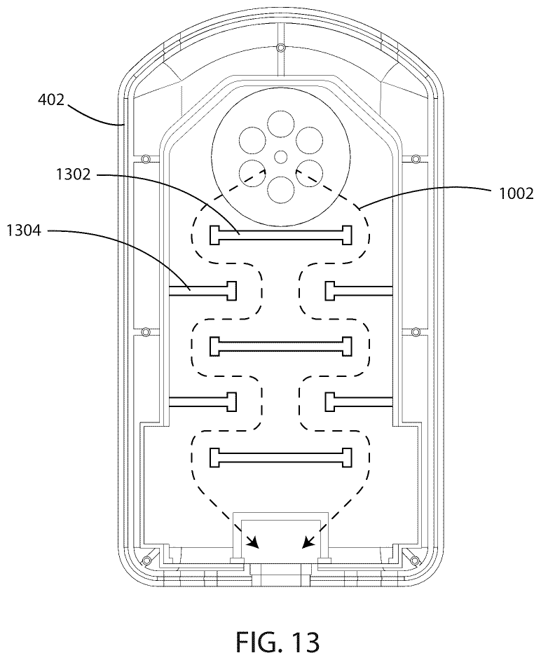

Referring now to FIG. 13, a schematic view of a piece 402 of a clam shell housing 102 is shown in accordance with various embodiments herein along with a flow path 1002 passing therethrough. In this embodiment, one or more center baffles 1302 (or flow guides) are included along with one or more side baffles 1304 (or flow guides). In various embodiments, the flow path 1002 includes a plurality of portions where the flow path 1002 moves laterally with respect to a lengthwise axis of the housing and/or the sensor board 408. In various embodiments, the flow path 1002 includes at least one segment that can be perpendicular to a lengthwise axis of the housing and/or the sensor board 408 and falls within a plane that can be parallel to a side of the sensor board 408. Beyond their role in defining flow paths, baffles herein can also provide additional surface area upon which to collect condensate that may occur.

In some embodiments, graphene sensors herein can be sealed off from the environment until the system is ready to accept a gas sample. For example, in some embodiments, graphene sensors herein can be bathed in an inert gas (such as nitrogen or the like) until the system is ready to accept a gas sample. In some embodiments, a sealing layer (such as a plastic layer or a metal foil layer) can initially be disposed over the array of graphene sensors, such as being placed there at the time of manufacture. Then later, the sealing layer can be removed. For example, a tab connected to the sealing layer can be pulled in order to remove the sealing layer and expose the array of graphene sensors.

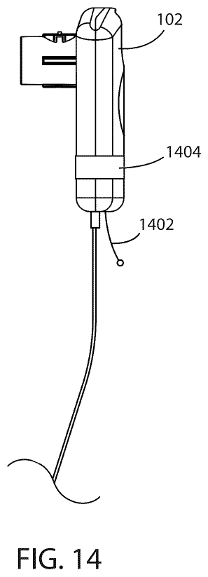

Referring now to FIG. 14, a schematic view of components of a gas sample evaluation system for gas samples is shown in accordance with various embodiments herein. As before, the measurement system for gas samples includes a housing 102. In this case, the measurement system includes a removable hermetic sealing layer (not visible in this view) and a pull tab 1402. Pulling on the pull tab 1402 can cause the sealing layer to release from the sensor board and thereby expose the array of graphene sensors.

In some cases, it can be desirable to cause the two pieces of the clam shell housing to be secured together in such a manner that any tampering will become evident. In some embodiments, the measurement system for gas samples also includes a security seal 1404. The security seal 1404 can be configured to maintain engagement of the pieces of a two-piece clam shell 202. In some embodiments, the security seal 1404 can be frangible to make disengagement and reengagement of the pieces of the two-piece clam shell 202 plainly visible.

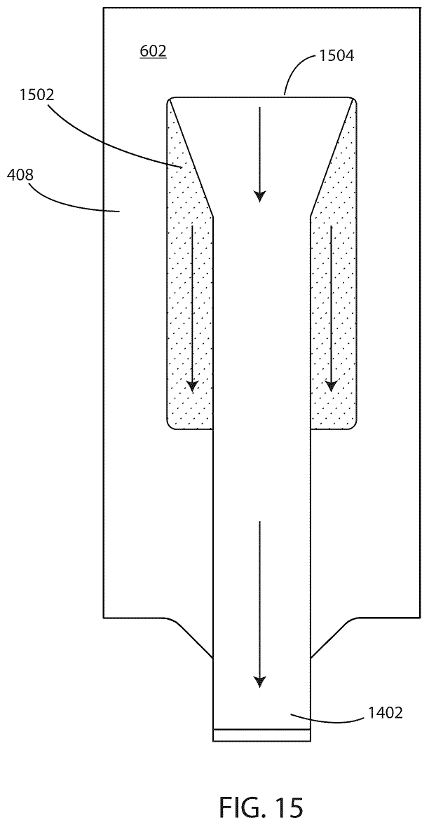

Referring now to FIG. 15, a schematic view of a side 602 of a sensor board 408 is shown in accordance with various embodiments herein. In this view, a removable hermetic sealing layer 1502 is shown which is disposed over the plurality of graphene sensors. In various embodiments, the removable hermetic sealing layer 1502 seals in an inert gas against a plurality of graphene sensors 608. The removable hermetic sealing layer 1502 also includes a pull tab 1402. The system can be configured so that when the pull tab 1402 is pulled by a system user, the removable hermetic sealing layer 1502 will be released, exposing the array of graphene sensors at the time that the device software is ready to collect measurement data. In specific, pulling on pull tab 1402 can exert a force on the edge 1504 of the portion of the removeable hermetic sealing layer 1502 that is bound down to the sensor board 408, which can cause the removeable hermetic sealing layer 1502 to begin peeling away from the sensor board 408.

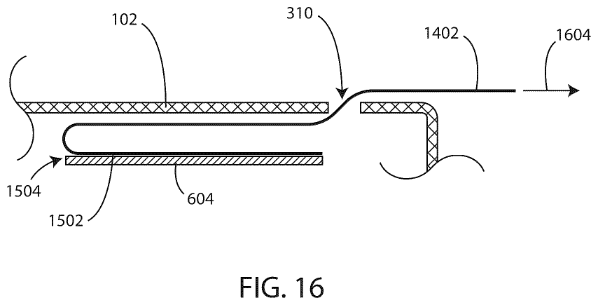

Referring now to FIG. 16, a schematic sectional view of a portion of a device herein illustrating how a removable hermetic sealing layer can be removed. In this view, the removable hermetic sealing layer 1502 can be seen disposed on/over sensor area 604, and thereby sealing in the sensors thereon (not shown in this view). The housing 102 can include a slot 310 through which the pull tab 1402 extends. In operation, the pull tab 1402 can be pulled in the direction of arrow 1604. Then, the edge 1504 of the removeable hermetic sealing layer 1502 can begin peeling back and continue until it is fully removed exposing sensor area 604 and the sensors thereon.

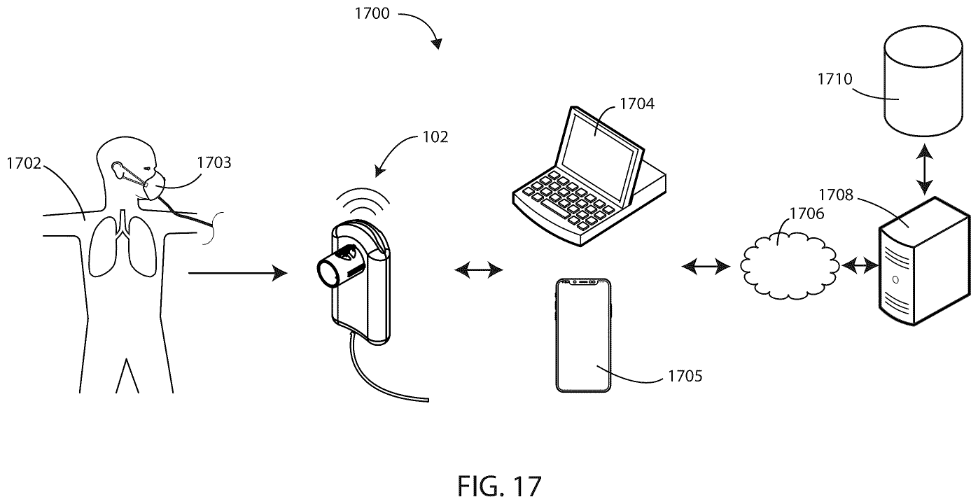

Systems herein can include various components and/or exchange data with various components. Referring now to FIG. 17, a schematic view of components of a system 1700 for measuring analyte presence in a gaseous sample is shown in accordance with various embodiments herein. The system 1700 can be configured to receive a gas sample from a subject 1702. The system 1700 can include main sensor housing 102 that includes an array of graphene sensors for sensing analytes in a gaseous mixture. In some embodiments, the graphene sensors can include graphene-based variable capacitors (or graphene varactors), as will be described in more detail below. The terms “discrete binding detector” and “graphene sensor” can be used interchangeably herein unless otherwise specified or the context dictates otherwise. The gas sample from the subject 1702 can be delivered to the main sensor housing 102 (directly or indirectly). For example, in some embodiments, the subject 1702 can wear a gas collection mask 1703 which can be used to gather and convey a gas sample onto the main sensor housing 102, but in other embodiments a mask is omitted.

In the embodiment shown in FIG. 17, the main sensor housing 102 of system 1700 is depicted in a hand-held format that can be used in the field, medical clinic, workplace, veterinary setting, and the like. It will be appreciated that the system herein can further include a table-top system for use in a medical clinic, hospital, laboratory, etc. However, it will be appreciated that many other formats for the main sensor housing 102 and system 1700 are also contemplated herein.

In some embodiments, the system 1700 can include and/or communicate with a local computing device 1704 that can include a microprocessor, input and output circuits, input devices, a visual display, a user interface, and the like. In some embodiments, the main sensor housing 102 can communicate with the local computing device 1704 to exchange data between the main sensor housing 102 and the local computing device 1704. The local computing device 1704 can be configured to perform various processing steps with the data received from the main sensor housing 102, including, but not limited to, calculating various parameters of the graphene varactors described herein. However, it should be appreciated that in some embodiments the features associated with the local computing device 1704 can be integrated into the main sensor housing 102. In some embodiments, the local computing device 1704 can be a laptop computer, a desktop computer, a server (real or virtual), a purpose dedicated computer device, or a portable computing device (including, but not limited to, a tablet, wearable device, etc.). The local computing device 1704 and/or the main sensor housing 102 can communicate with computing devices in remote locations through a data network 1706, such as the Internet or another network for the exchange of data as packets, frames, or otherwise. In some embodiments, a smart phone 1705 or mobile phone can be included and take on the functions described herein with respect to the local computing device 1704.

In some cases, various operations can be performed in order to facilitate processing at the edge (e.g., with the main sensor housing 102 and/or a local computing device 1704). By way of example, in some cases template patterns used herein or other data can be saved in memory by the main sensor housing 102 and/or a local computing device 1704 for use in executing operations herein. In some embodiments, some data can be discarded or otherwise not used in order to simplify calculations herein.

However, in some embodiments, various operations herein can be performed, at least in part, by remote computing resources. For example, in some embodiments, the system 1700 can also include a computing device such as a server 1708 (real or virtual). In some embodiments, the server 1708 can be located remotely from the main sensor housing 102. The server 1708 can be in data communication with a database 1710. The database 1710 can be used to store various subject information, such as that described herein. In some embodiments, the database 1710 can specifically include characteristic patterns of data (or templates) and the like.

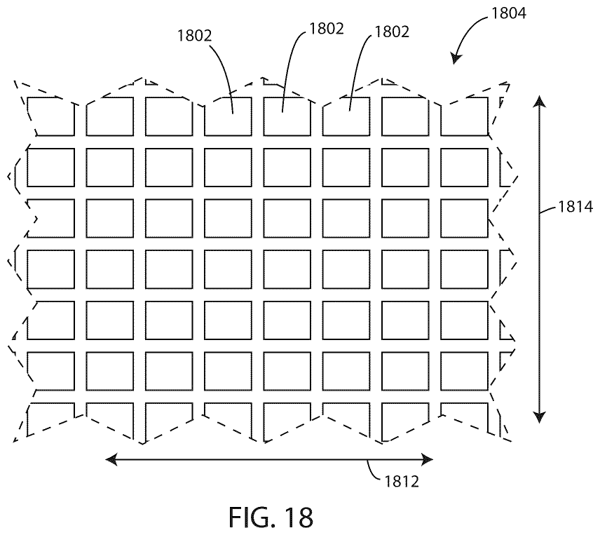

Each chemical sensor element herein can include one or more discrete binding detectors in an array throughout the measurement zones. Referring now to FIG. 18, a schematic diagram of a portion of a chemical sensor element is shown in accordance with various embodiments herein. A plurality of graphene varactors 1802 can be disposed on the first measurement zone 1804 in an array within the measurement zones. In some embodiments, a chemical sensor element can include a plurality of graphene varactors configured in an array. In some embodiments, the plurality of graphene varactors can include identical surface chemistries, while in other embodiments the plurality of graphene varactors can include different surface chemistries from one another. In some embodiments, graphene varactors having the same surface chemistries can be present in duplicate, triplicate, or more, such that data obtained during measurement cycles can be averaged together to further refine the change observed in the response signals. The graphene varactors herein can be as described in more detail in U.S. Pat. No. 9,513,244, which is herein incorporated by reference in its entirety. It will be appreciated that any of the first measurement zone, the second measurement zone, the third measurement zone, and the like can include one or more arrays of a plurality of graphene varactors as described herein.

In some embodiments, the graphene varactors can be heterogeneous in that they are different (in groups or as individual graphene varactors) from one another in terms of their binding behavior or specificity with regard to a particular analyte. In some embodiments, some graphene varactors can be duplicated, triplicated, or more, for validation purposes but are otherwise heterogeneous from other graphene varactors. Yet in other embodiments, the graphene varactors can be homogeneous. While the graphene varactors 1802 of FIG. 18 are shown as boxes organized into a grid, it will be appreciated that the graphene varactors can take on many different shapes (including, but not limited to, various polygons, circles, ovals, irregular shapes, and the like) and, in turn, the groups of graphene varactors can be arranged into many different patterns (including, but not limited to, star patterns, zig-zag patterns, radial patterns, symbolic patterns, and the like).

In some embodiments, the order of specific graphene varactors 1802 across a length 1812 and width 1814 of the measurement zone can be substantially random. In other embodiments, the order can be specific. For example, in some embodiments, a measurement zone can be ordered so that the specific graphene varactors 1802 configured to bind to analytes having a lower molecular weight are located farther away from the incoming gas flow relative to specific graphene varactors 1802 configured to bind to analytes having a higher molecular weight which are located closer to the incoming gas flow. As such, chromatographic effects which may serve to provide separation between chemical compounds of different molecular weight can be taken advantage of to provide for optimal binding of chemical compounds to corresponding graphene varactors.

The number of graphene varactors can be from about 1 to about 100,000. In some embodiments, the number of graphene varactors can be from about 1 to about 10,000. In some embodiments, the number of graphene varactors can be from about 1 to about 1,000. In some embodiments, the number of graphene varactors can be from about 2 to about 500. In some embodiments, the number of graphene varactors can be from about 10 to about 500. In some embodiments, the number of graphene varactors can be from about 50 to about 500. In some embodiments, the number of graphene varactors can be from about 1 to about 250. In some embodiments, the number of graphene varactors can be from about 1 to about 50.

The graphene varactor or other graphene sensor herein can include a graphene layer and a self-assembled monolayer disposed on an outer surface of the graphene layer through x-x stacking interactions. Graphene is a form of carbon containing a single layer of carbon atoms in a hexagonal lattice. Graphene has a high strength and stability due to its tightly packed sp2 hybridized orbitals, where each carbon atom forms one sigma (σ) bond each with its three neighboring carbon atoms and has one p orbital projected out of the hexagonal plane. The p orbitals of the hexagonal lattice can hybridize to form a π bond suitable for non-covalent π-π stacking interactions with other π-electron rich molecules.

The non-covalent functionalization of graphene with a self-assembled monolayer does not significantly affect the atomic structure of graphene, and provides a stable graphene-based sensor with high sensitivity towards a number of volatile organic compounds (VOCs) in the parts-per-billion (ppb) or parts-per-million (ppm) levels. The use of specific compounds with which to form a self-assembled monolayer on the graphene can increase the sensitivity towards target compounds and/or metabolites thereof allowing for collection of a richer dataset.

As such, the graphene varactors described herein can include those in which a single graphene layer has been surface-modified through non-covalent π-π stacking interactions between graphene and π-electron-rich molecules, such as, for example, at least one of pyrenes, coronenes, aromatic cyclodextrins, and pillarenes. In some embodiments, at least two of pyrenes, coronenes, aromatic cyclodextrins, and pillarenes are included on graphene surfaces herein. In some embodiments, at least three of pyrenes, coronenes, aromatic cyclodextrins, and pillarenes are included on graphene surfaces herein. In some embodiments, at least four of pyrenes, coronenes, aromatic cyclodextrins, and pillarenes are included on graphene surfaces herein. Aromatic cyclodextrins can include benzylated cyclodextrins, such as α-, β- and γ-cyclodextrins derivatives, including, but not limited to, α-CDBn18, β-CDBn21, γ-CDBn24, β-CDBn19(OH)2.

In some embodiments, the self-assembled monolayer can provide at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% surface coverage (by area) of the graphene layer. It will be appreciated that the self-assembled monolayer can provide surface coverage falling within a range wherein any of the forgoing percentages can serve as the lower or upper bound of the range, provided that the lower bound of the range is a value less than the upper bound of the range.

In some embodiments, it will be appreciated that the self-assembly of π-electron-rich molecules on the surface of the graphene layer can include the self-assembly into more than a monolayer, such as a multilayer. Multilayers can be detected and quantified by techniques such as scanning tunneling microscopy (STM) and other scanning probe microscopies. References herein to a percentage of coverage greater than 100% shall refer to the circumstance where a portion of the surface area is covered by more than a monolayer, such as covered by two, three or potentially more layers of the compound used. Thus, a reference to 105% coverage herein shall indicate that approximately 5% of the surface area includes more than monolayer coverage over the graphene layer. In some embodiments, graphene surfaces can include 101%, 102%, 103%, 104%, 105%, 110%, 120%, 130%, 140%, 150%, or 175% surface coverage of the graphene layer. It will be appreciated that multilayer surface coverage of the graphene layer can fall within a range of surface coverages, wherein any of the forgoing percentages can serve as the lower or upper bound of the range, provided that the lower bound of the range is a value less than the upper bound of the range. For example, ranges of coverage can include, but are not limited to, 50% to 150% by surface area, 80% to 120% by surface area, 90% to 110%, or 99% to 120% by surface area.

In some embodiments, the self-assembled monolayers suitable for use herein can provide coverage of the graphene surface with a monolayer as quantified by the Langmuir theta value of at least some minimum threshold value, but avoid covering the majority of the surface of the graphene with a multilayer thicker than a monolayer. In some embodiments, the self-assembled monolayers suitable for use herein provide a Langmuir theta value of at least 0.95. In some embodiments, the self-assembled monolayers suitable for use herein provide a Langmuir theta value of at least 0.98. In some embodiments, the self-assembled monolayers can provide a Langmuir theta value of at least 0.85, 0.86, 0.87, 0.88, 0.89, 0.90, 0.91, 0.92, 0.93, 0.94, 0.95, 0.96, 0.97, 0.98, 0.99, or 1.0. It will be appreciated that the self-assembled monolayer can provide a range of Langmuir theta values, wherein any of the forgoing Langmuir theta values can serve as the lower or upper bound of the range, provided that the lower bound of the range is a value less than the upper bound of the range.

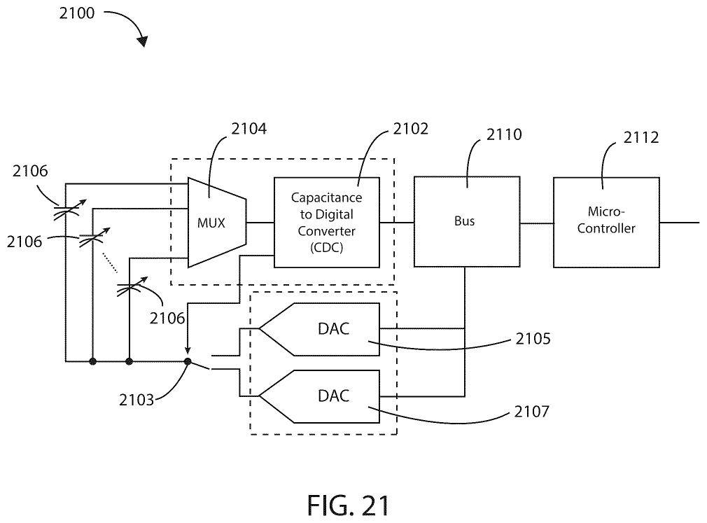

In some embodiments, each of the graphene varactors suitable for use herein can include at least a portion of one or more electrical circuits. By way of example, in some embodiments, each of the graphene varactors can include all or a portion of one or more passive electrical circuits or active electrical circuits. In some embodiments, the graphene varactors herein can include two-terminal graphene varactors. In some embodiments, the two-terminal graphene varactors can be adapted to each receive independent signals from an electrical signal generator. In some embodiments, the graphene varactors can be formed such that they are integrated directly on an electronic circuit. In some embodiments, the graphene varactors can be formed such that they are wafer bonded to the circuit. In some embodiments, the graphene varactors can include integrated readout electronics, such as a readout integrated circuit (ROIC). The electrical properties of the electrical circuit, including resistance or capacitance, can change upon binding, such as specific and/or non-specific binding, with a compound from a biological sample. Many different types of circuits can be used to gather data from chemical sensor elements and will be discussed below in reference to FIG. 21.

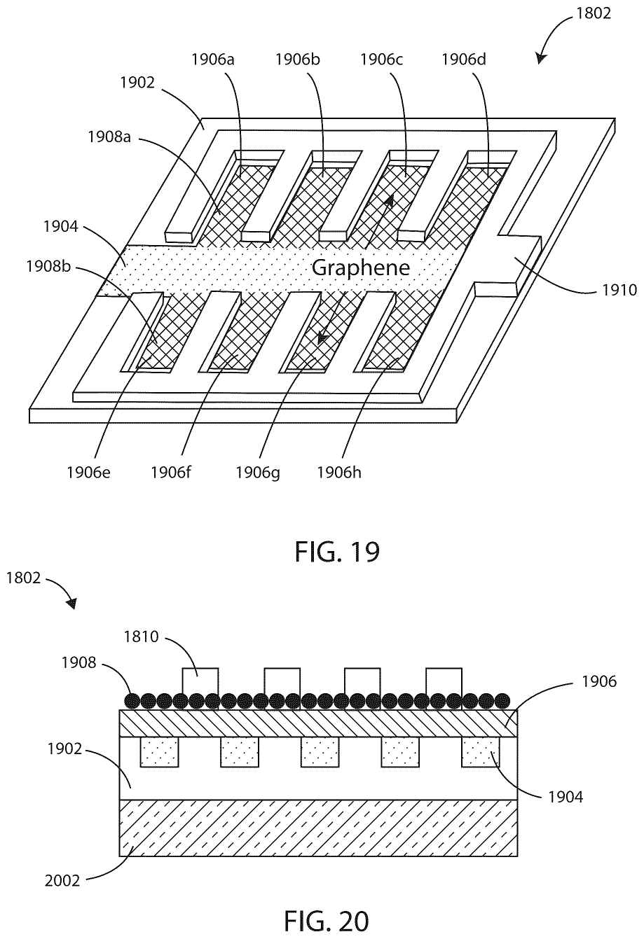

Referring now to FIG. 19, a schematic view of a graphene varactor 1802 having two terminals is shown in accordance with the embodiments herein. It will be appreciated that graphene varactors can be prepared in various ways with various geometries, and that the graphene varactor shown in FIG. 19 is just one example in accordance with the embodiments herein.

Graphene varactor 1802 can include an insulator layer 1902, a gate electrode 1904 (or “gate contact”), a dielectric layer (item 1906 in FIG. 20), one or more graphene layers, such as graphene layers 1908a and 1908b, and a contact electrode 1910 (or “graphene contact”). In some embodiments, the graphene layer(s) 1908a-b can be contiguous, while in other embodiments the graphene layer(s) 1908a-b can be non-contiguous. Gate electrode 1904 can be deposited within one or more depressions formed in insulator layer 1902. Insulator layer 1902 can be formed from an insulative material such as silicon dioxide, formed on a substrate (item 2002 in FIG. 20), such as a silicon substrate (wafer), and the like. Gate electrode 1904 can be formed by an electrically conductive material such as chromium, copper, gold, silver, tungsten, aluminum, titanium, palladium, platinum, iridium, and any combinations or alloys thereof, which can be deposited on top of or embedded within the insulator layer 1902. The dielectric layer (not shown in FIG. 19) can be disposed on a surface of the insulator layer 1902 and the gate electrode 1904, as shown in more detail in FIG. 20. The graphene layer(s) 1908a-b can be disposed on the dielectric layer. Various layer configurations, thicknesses and electrical connections may be included.

Graphene varactor 1802 includes eight gate electrode fingers 1906a-1906h. It will be appreciated that while graphene varactor 1802 shows eight gate electrode fingers 1906a-1906h, any number of gate electrode finger configurations can be contemplated. In some embodiments, an individual graphene varactor can include fewer than eight gate electrode fingers. In some embodiments, an individual graphene varactor can include more than eight gate electrode fingers. In other embodiments, an individual graphene varactor can include two gate electrode fingers. In some embodiments, an individual graphene varactor can include 1, 2, 3, 4, 5, 6, 7, 8, 9, 10, or more gate electrode fingers.

Graphene varactor 1802 can include one or more contact electrodes 1910 disposed on portions of the graphene layers 1908a and 1908b. Contact electrode 1910 can be formed from an electrically conductive material such as chromium, copper, gold, silver, tungsten, aluminum, titanium, palladium, platinum, iridium, and any combinations or alloys thereof. Further aspects of exemplary graphene varactors can be found in U.S. Pat. No. 9,513,244, the content of which is herein incorporated by reference in its entirety.

Referring now to FIG. 20, a schematic diagram of a portion of a cross-sectional view of an exemplary graphene varactor is shown in accordance with various embodiments herein. The graphene varactor can include a substrate 2002, such as a silicon substrate (wafer). An insulator layer 1902 can be disposed on the substrate 2002, and a gate electrode 1904 can be recessed into the insulator layer 1902. The gate electrode 1904 can be formed by depositing an electrically conductive material in the depression in the insulator layer 1902. A dielectric layer 1906 can be formed on a surface of the insulator layer 1902 and the gate electrode 1904. In some examples, the dielectric layer 1906 can be formed of a material, such as, silicon dioxide, aluminum oxide, hafnium dioxide, zirconium dioxide, hafnium silicate, or zirconium silicate. The graphene layer 1908 is disposed on the dielectric layer 1906 and the contact electrode 1910 can be disposed in contact with the graphene layer 1908. In some examples, the dielectric layer 1906 can include multiple layers of the dielectric materials listed herein. In some embodiments, the dielectric layer 1906 can include alternating layers of different dielectric materials. In some embodiments, the dielectric layer 1906 can include alternating layers of aluminum oxide and hafnium dioxide.

In some embodiments herein, to maintain the stability of the graphene varactors herein, the chemical sensor elements can be pretreated under a vacuum at a temperature from 50° C. to 150° C. for at least 3 hours. In various embodiments, the chemical sensor elements can be pretreated under vacuum at a temperature from 120° C. to 150° C. for 10 to 20 hours. In various embodiments, the chemical sensor elements can be pretreated under a vacuum at a temperature can be greater than or equal to 50° C., 60° C., 70° C., 80° C., 90° C., 100° C., 110° C., 120° C., 130° C., 140° C., 150° C., 160° C., 170° C., 180° C., 190° C., or 200° C., or can be an amount falling within a range between any of the foregoing.

In addition, the chemical sensor elements herein can be maintained under a controlled gas environment until it is exposed to a gaseous test sample. By way of example, the chemical sensor element can be maintained under a controlled gas environment including oxygen gas, nitrogen gas or an inert gas such as, for example, argon, helium, xenon, krypton, or neon.

In many cases, each graphene varactor can be interrogated using a number of different applied voltages (a plurality of voltages) with the resulting data forming a C-V_{g}curve. The plurality of voltages can fall within a range from a lower voltage bound to an upper voltage bound. In many cases the voltages may start at the lower bound and then be increased progressing up to the upper bound, thus sweeping across the range in a first direction followed by a sweep in the opposite (or second) direction (e.g., from the upper bound to the lower bound). Thus, in various embodiments, a first direction can include a sweep from the lower voltage bound to the upper voltage bound and a second direction is a sweep from the upper voltage bound to the lower voltage bound. However, in other embodiments, the first direction can include a sweep from the upper voltage bound to the lower voltage bound and the second direction is a sweep from the lower voltage bound to the upper voltage bound. In various embodiments, a sweep in the first direction followed by a sweep in the second direction constitutes a measurement cycle.

The values for the lower voltage bound and the upper voltage bounds can be predetermined or can be determined dynamically. In various embodiments, the lower voltage bound and the upper voltage bound are preset values and can be selected from values such as −3.0 V or less, −2.9 V, −2.8 V, −2.7 V, −2.6 V, −2.5 V, −2.4 V, −2.3 V, −2.2 V, −2.1 V, −2.0 V, −1.9 V, −1.8 V, −1.7 V, −1.6 V, −1.5 V, −1.4V, −1.3 V, −1.2 V, −1.1 V, −1.0 V, −0.9 V, −0.8 V, −0.7 V, −0.6 V, −0.5 V, −0.4V, −0.3 V, −0.2 V, −0.1 V, 0V, 0.1V, 0.2 V, 0.3 V, 0.4 V, 0.5 V, 0.6 V, 0.7V, 0.8 V, 0.9V, 1.0V, 1.1 V, 1.2V, 1.3 V, 1.4V, 1.5 V, 1.6 V, 1.7 V, 1.8 V, 1.9 V, 2.0 V, 2.1 V, 2.2 V, 2.3 V, 2.4 V. 2.5 V, 2.6 V, 2.7 V, 2.8 V, 2.9 V, or 3.0 V or more, or a voltage value falling between any of the foregoing values. In various embodiments, the lower voltage bound and the upper voltage bound are preset values and can be selected from values ranging from −5 V to 5 V; from −4 V to 4 V; from −3 V to 3 V; from −2 V to 2 V; from −1.5 V to 1.5V; or from −1 V to 1 V.

While an instantaneous applied voltage herein can be thought of as the sum of a DC bias component and an AC component, it will be appreciated that specific applied voltages values as referenced herein typically represent the DC voltage bias or offset value. This is because the average value of an AC component over a non-instantaneous time period will be zero. As such, unless otherwise stated to the contrary or the context dictates otherwise, voltage value references herein shall refer to the DC bias or offset component of an applied voltage, understanding that corresponding instantaneous voltage values can vary based on the AC component. The waveforms of the AC component can take many different forms. For example, they can be sinusoidal, square, triangular, trapezoidal, ramped, sawtooth, complex, or the like.

In some embodiments, the lower voltage bound and the upper voltage bound are dynamically determined values. For example, the bounds can be changed based on previously applied excitation voltages and/or previously observed values related to the graphene sensor and/or previously observed effects.

In some embodiments the upper voltage bound and the lower voltage bound is static between successive measurement cycles. In other embodiments, the upper voltage bound and the lower voltage bound may change between successive measurement cycles. For example, in some embodiments, the first measurement cycle can include the use of the widest range of excitation voltages and successive measurement cycles may utilize a narrower range of excitation voltages.

Various timing schemes can be used for the sweep across a range of voltages. In some embodiments, a sweep in the first direction can be immediately followed by a sweep in the second direction. In other embodiments, a sweep in the first direction can be followed by a pause and then a sweep in the second direction. The duration of a pause between sweeps can include those from 1 millisecond (ms) to 5 seconds in length. In some embodiments, the duration of the pause between sweeps can be greater than or equal to, 10 ms, 20 ms, 30 ms, 40 ms, 50 ms, 60 ms, 70 ms, 80 ms, 90 ms, 100 ms, 200 ms, 300 ms, 400 ms, 500 ms, 600 ms, 700 ms, 800 ms, 900 ms, or 1 sec, 2 sec, 3 sec, 4 sec, or 5 sec, or can be an amount falling within a range between any of the foregoing.

In some embodiments, the duration of a pause between sweeps can be greater than 5 seconds in length. In various embodiments, the duration of a pause between sweeps can greater than or equal to 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, or 60 sec, or can be an amount falling within a range between any of the foregoing. In other embodiments, the duration of a pause between sweeps can be greater than 1 minute.

A change in any one of the parameters of the capacitance versus voltage values provides data that can reflect the binding status of analytes to the graphene varactor(s) and can be used to characterize a sample and/or distinguish various analytes and analyte concentrations in the sample.

Various measurable aspects can be used to characterize the content of a sample (such as a breath sample). In some embodiments, a ratio of the maximum capacitance to minimum capacitance can be used to characterize the content of a gaseous mixture. In some embodiments, a ratio of the maximum capacitance to the shift in the Dirac point can be used to characterize the content of a gaseous mixture. In other embodiments, a ratio of the minimum capacitance to the shift in the slope of the response signal can be used to characterize the content of a gaseous mixture. In some embodiments, a ratio of any of the parameters including a shift in the Dirac point, a change in the minimum capacitance, a change in the slope of the response signal, or the change in the maximum capacitance can be used to characterize the content of a sample mixture. In accordance with embodiments herein, hysteresis effects observed with respect to any of these values (as well as other types of values discussed) can be used to characterize the content of sample mixtures.

Various measurement circuitry can be used to measure the changes in the parameters of the capacitance-voltage curve of the graphene varactor(s). Measurement circuitry suitable for use herein can include active and passive sensing circuits. Such circuitry can implement wired (direct electrical contact) or wireless sensing techniques.

Measurement circuitry herein can also include active sensing circuits. In various embodiments, the measurement circuity can include an electrical signal generator configured to generate a series of measurement cycles over a time period. The measurement circuity can include an electrical signal generator configured to generate and deliver an applied voltage that can be represented as an alternating voltage (or excitation voltage) superimposed on a bias voltage. It will be appreciated that there are many ways to generate such an applied voltage.

In some embodiments, measurement circuity can include an electrical signal generator configured to generate and deliver an applied voltage that includes a sinusoidal, square, triangular, trapezoidal, ramped, sawtooth, or complex waveform alternating voltage superimposed on a bias voltage. In some embodiments, the electrical signal generator can be configured to generate an applied voltage at a plurality of voltages to be applied to the one or more graphene varactors, the voltages falling within a range from a lower voltage bound and an upper voltage bound, the voltages starting at one bound and moving to the other bound as part of a sweep across the voltages. In some embodiments, the electrical signal generator can be configured to generate an excitation current at a plurality of voltages to be applied to the one or more graphene varactors, the voltages falling within a range from a lower bound and an upper bound, the voltages starting at one bound and moving to the other bound as part of a sweep across the voltages.

Referring now to FIG. 21, a schematic diagram is shown of an example of measurement circuity 2100 to measure the capacitance of a plurality of graphene sensors in accordance with various embodiments herein. The measurement circuity 2100 can include a capacitance to digital converter (CDC) 2102 in electrical communication with a multiplexor 2104. The multiplexor 2104 can provide selective electrical communication with a plurality of graphene varactors 2106. The connection to the other side of the graphene varactors 2106 can be controlled by a switch 2103 (as controlled by the CDC) and can provide selective electrical communication with a first digital to analog converter (DAC) 2105 and a second digital to analog converter (DAC) 2107. The other side of the DACs 2105, 2107 can be connected to a bus device 2110, or in some cases, the CDC 2102. The circuitry can further include a microcontroller 2112 (or controller circuit), which will be discussed in more detail below.

In this case, a signal from the CDC controls the switch 2103 between the output voltages of the two programmable Digital to Analog Converters (DACs) 2105 and 2107. The programmed voltage difference between the DACs determines an excitation amplitude (and represents the AC component of the applied voltage), providing an additional programmable scale factor to the measurement and allowing measurement of a wider range of capacitances than specified by the CDC. The bias voltage at which the capacitance is measured is equal to the difference between the bias voltage at the CDC input (via the multiplexor, usually equal to VCC/2, where VCC is the supply voltage) and the average voltage of the excitation signal, which is programmable. In some embodiments, buffer amplifiers and/or bypass capacitance can be used at the DAC outputs to maintain stable voltages during switching. It will be appreciated that the circuits of FIG. 21 are merely exemplary. Many different approaches are contemplated herein.

The measurement circuity can include a capacitance sensor configured to measure capacitance of the discrete binding detectors resulting from the excitation voltage. The measurement circuity can also include a controller circuit configured to determine a change in at least one of a measured capacitance versus voltage value and a calculated value based on the measured capacitance or voltage over the time period. In various embodiments, the measured capacitance versus voltage values can include one or more of a capacitance at a particular voltage, a maximum slope of capacitance to voltage, a minimum slope of capacitance to voltage, a minimum capacitance, a voltage at minimum capacitance (Dirac voltage), a maximum capacitance, and a ratio of maximum capacitance to minimum capacitance. In various embodiments, the controller circuit is configured to measure a difference between a forward Dirac point voltage and a reverse Dirac point voltage. In some embodiments, the controller circuit is configured to calculate a rate of change of measured capacitance over the time period at multiple discrete DC bias voltages. In some embodiments, the controller circuit is configured to calculate an average hysteresis change value of a measured property over a plurality of measurement cycles. In various embodiments, the controller circuit is configured to determine the forward Dirac point voltage and/or the reverse Dirac point voltage.

In some embodiments, the measurement circuitry or another part of the system herein can include a temperature controller configured to control a temperature of the graphene varactors. In some embodiments, the temperature controller can include a thermistor, thermocouple, resistive thermal device (RTD) and the like. In various embodiments, controlling the temperature of the graphene varactors comprises exposing the graphene varactor to one or more temperature set points for a predetermined time. In some embodiments, a sequence involving increasing the temperature set points over a course of a predetermined time can be used. In other embodiments, a sequence involving decreasing the temperature set points over a course of a predetermined time can be used. In other embodiments, a sequence involving increasing the temperature set points followed by decreasing the temperature set points can be used. Configuration parameter settings can be modified in the operating software for various use cases.

The system for measuring analyte presence in a gaseous sample can be configured to measure differences in a capacitance versus voltage value when an applied voltage is swept in a first direction between the lower voltage bound and upper voltage bound versus a second direction between the between the upper voltage bound and lower voltage bound. In various embodiments, the first direction is a sweep from the lower voltage bound to the upper voltage bound and the second direction is a sweep from the upper voltage bound to the lower voltage bound. In various embodiments, the first direction is a sweep from the upper voltage bound to the lower voltage bound and the second direction is a sweep from the lower voltage bound to the upper voltage bound.

Various values for the voltages suitable for use within a range from a lower bound to an upper bound as contemplated herein are described further below. In various embodiments, each measurement cycle includes delivering a DC bias voltage to the discrete binding detectors at multiple discrete DC bias voltage values across a range of DC bias voltages as discussed in greater detail below.

In some cases, the above calculated values can be indicative of the identity and/or concentrations of specific volatile organic components of a gas sample. As such, each of the calculated values above can serve as a distinct piece of data that forms part of a pattern for a given subject and/or given gas sample. As also described elsewhere herein, the pattern can then be matched against preexisting patterns, or patterns identified in real-time, derived from large, stored data sets through techniques such as machine learning or other techniques, wherein such patterns are determined to be characteristic of specific disease states, health statuses, or the like. The above calculated aspects can also be put to other purposes, diagnostic and otherwise.

In some embodiments, calculations such as those described above can be performed by a controller circuit. The controller circuit can be configured to receive an electrical signal reflecting the capacitance or voltage of the graphene varactors. In some embodiments, the controller circuit can include a microcontroller to perform these calculations. In some embodiments, the controller circuit can include a microprocessor in electrical communication with the measurement circuity. The microprocessor system can include components such as an address bus, a data bus, a control bus, a clock, a CPU, a processing device, an address decoder, RAM, ROM and the like. In some embodiments, the controller circuit can include a calculation circuit (such as an application specific integrated circuit-ASIC) in electrical communication with the measurement circuity.

In addition, in some embodiments, the system can include a nonvolatile memory. In some embodiments, the non-volatile memory can be configured to store measured capacitance values for the discrete binding detectors across a range of DC bias voltages. In other embodiments, the nonvolatile memory can be configured to store a baseline capacitance for the discrete binding detectors across a range of DC bias voltages. In some embodiments, the nonvolatile memory can be where sensitivity calibration information for the graphene varactors is stored.

By way of example, the graphene varactors can be tested in a production facility, where sensitivity to various analytes such as VOC's can be determined and then stored on an EPROM or similar component. In addition, or alternatively, sensitivity calibration information can be stored in a central database and referenced with a chemical sensor element serial number when subject data is sent to a central location for analysis and diagnosis. These components can be included with any of the pieces of hardware described herein.

In some embodiments herein, components can be configured to communicate over a network, such as the internet or a similar network. In various embodiments, a central storage and data processing facility can be included. In some embodiments, data gathered from sensors in the presence of the subject (local) can be sent to the central processing facility (remote) via the internet or a similar network, and the pattern from the particular subject being evaluated can be compared against pre-stored patterns.

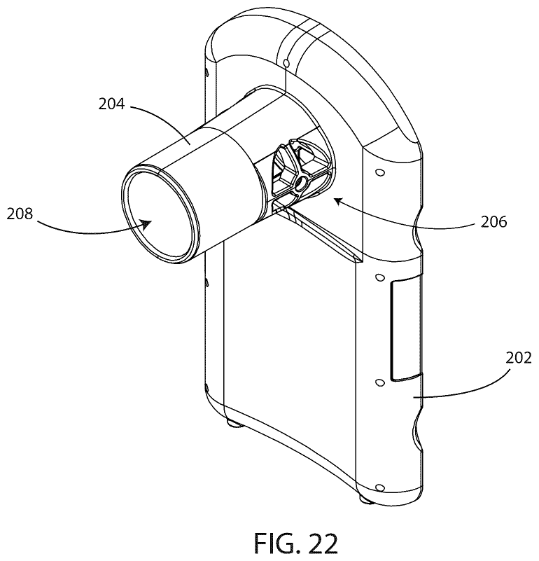

It will be appreciated that, in some embodiments, some alternative configurations can be utilized. By way of example, FIG. 22 is a schematic perspective view of a portion of a gas sample evaluation system housing unit in accordance with various embodiments herein. FIG. 22 is similar to FIG. 2 and shows one piece of the two-piece clam shell 202 thereof. As such, FIG. 22 includes inflow tube 204, inspiration ports 206, and inflow port 208. However, FIG. 22 shows the inspiration ports 206 (one is visible, and one is not shown in this view) disposed on lateral opposed sides of the inflow tube 204 versus on top thereof. In some embodiments, inspiration ports 206 (for example, from one to four of them) can be disposed on the top, bottom, and/or sides of the inflow tube 204.

It will be appreciated that many different methods are contemplated herein, including, but not limited to, methods of making system components herein, methods of using system components herein, and the like. Aspects of system/device operation described elsewhere herein can be performed as operations of one or more methods in accordance with various embodiments herein.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A measurement system for gas samples comprising:
a housing, the housing;
defining an inflow port; and
defining an outflow port;
a sensor board, wherein the sensor board is disposed within the housing, the sensor board comprising
a first side; and
a second side;
the measurement system for gas samples defining a flow path, wherein the flow path extends from the inflow port to the outflow port; and
a plurality of graphene sensors, wherein the plurality of graphene sensors are disposed on the second side.

2. The measurement system for gas samples of claim 1, the sensor board further comprising a circuit board.

3. The measurement system for gas samples of claim 1, the graphene sensors comprising graphene varactors.

4. The measurement system for gas samples of claim 1, wherein the flow path passes along and is in contact with a lengthwise axis of the first side of the sensor board.

5. The measurement system for gas samples of claim 4, wherein the first side of the sensor board is configured to allow condensation of moisture thereon reducing the humidity of an incoming gas sample.

6. The measurement system for gas samples of claim 1, further comprising a control circuit, wherein the control circuit is configured to control operations of the measurement system for gas samples.

7. The measurement system for gas samples of claim 6, further comprising a power supply.

8. The measurement system for gas samples of claim 7, wherein the power supply is configured to receive 500 m (Original) A or less DC current.

9. The measurement system for gas samples of claim 6, further comprising measurement circuitry, wherein the measurement circuitry is configured to provide a stimulus voltage to the plurality of graphene sensors and measure an electrical property of the same.

10. The measurement system for gas samples of claim 6, further comprising wireless communications circuitry.

11. The measurement system for gas samples of claim 6, further comprising wired communications circuitry.

12. The measurement system for gas samples of claim 1, further comprising an inflow tube, wherein the inflow tube is in fluid communication with the inflow port.

13. The measurement system for gas samples of claim 12, the inflow tube comprising:
an inspiration port; and
an expiration port, wherein the expiration port is in fluid communication with the inflow port of the housing.

14. The measurement system for gas samples of claim 1, further comprising:
an inspiration valve, wherein the inspiration valve is a one-way valve controlling movement of air through an inspiration port; and
an expiration valve, wherein the expiration valve is a one-way valve controlling movement of air through the expiration valve.

15. The measurement system for gas samples of claim 1, further comprising an outflow valve, wherein the outflow valve is a one-way valve controlling movement of air through the outflow port.

16. The measurement system for gas samples of claim 1, further comprising at least one electrically powered heating element, wherein the at least one electrically powered heating element is disposed on the second side of the sensor board adjacent to the plurality of graphene sensors.

17. The measurement system for gas samples of claim 1, further comprising a removable hermetic sealing layer or other sealing materials, wherein the removable hermetic sealing layer or other sealing materials are disposed over the plurality of graphene sensors.

18. (canceled)

19. The measurement system for gas samples of claim 17, wherein the removable hermetic sealing layer or other sealing materials seals in an inert gas against the plurality of graphene sensors.

20. (canceled)

21. The measurement system for gas samples of claim 1, the housing comprising a two-piece clam shell.

22. The measurement system for gas samples of claim 21, further comprising a tamper-evident seal, wherein the tamper-evident seal is configured to maintain engagement of the pieces of the two-piece clam shell and the tamper-evident seal is frangible to make disengagement and reengagement of the pieces of the two-piece clam shell visible.
